(19) United States
(12) Patent Application Publication
(10) Pub. No.: US 2024/0181271 A1
(43) Pub. Date: Jun. 6, 2024

(54) APPARATUS AND METHOD FOR TREATING ACUTE CENTRAL NERVOUS SYSTEM INJURIES WITH PROMPT APPLICATION OF ELECTROMAGNETIC FIELDS

(71) Applicant: Weinberg Medical Physics, Inc., North Bethesda, MD (US)

(72) Inventor: Irving N. WEINBERG, Chevy Chase, MD (US)

(21) Appl. No.: 18/389,437

(22) Filed: Nov. 14, 2023

Related U.S. Application Data
(60) Provisional application No. 63/429,723, filed on Dec. 2, 2022.

(51) Int. Cl. A61N 2/00; A61N 2/06

(52) U.S. Cl. CPC A61N 2/006; A61N 2/06

(57) ABSTRACT

An apparatus and method are provided utilizing electropermanent magnets (EPMs) or other electromagnetic effectors to deliver either or both electrical and changing magnetic fields to rescue the brains or other sections of the nervous system of human or non-human animals that have been subject to acute injury.

### CROSS-REFERENCE AND PRIORITY CLAIM

This application claims priority to U.S. Provisional Patent Application Ser. No. 63/429,723, entitled “APPARATUS AND METHOD FOR TREATING ACUTE CENTRAL NERVOUS SYSTEM INJURIES WITH PROMPT APPLICATION OF ELECTROMAGNETIC FIELDS” filed Nov. 14, 2022, the entirety of which is incorporated by reference.

### FIELD

A method and apparatus for diagnosing and treating diseases in animals, including humans. The method and apparatus may be used in medical and veterinary applications.

### BACKGROUND

It is known that repair cells (oligodendrocyte precursors) migrate to the site of a brain stroke in animals within 72 hours after the initiation of a stroke (as taught by E. Bonifanti et al in the Cell Death and Disease publication of 2017 entitled: “The role of oligodendrocyte precursor cells after expressing the GPR17 receptor in brain remodeling after stroke”).

It is also known that a one-hour period of low-frequency electrical stimulation after injury to a nerve promotes eventual recovery, possibly due to prevention of irreversible denervation atrophy (as taught by T. Gordon et al in the Experimental Neurology publication of 2010 entitled “Brief post-surgical electrical stimulation accelerates axon regeneration and muscle reinnervation without affecting the functional measures in carpal tunnel syndrome patients”.

### SUMMARY

Disclosed embodiments detail the design and use of a system capable of delivering electrical and/or magnetic fields for neuromodulation and/or delivery of therapeutic compounds in the central nervous systems of humans or non-human animals who have undergone stroke or other brain damage.

An apparatus and method utilizing electropermanent magnets (EPMs) or other electromagnetic effectors to deliver either or both electrical and changing magnetic fields to rescue the brains (or other sections of the nervous system) of human or non-human animals that have been subject to acute injury.

### BRIEF DESCRIPTION OF THE FIGURES

FIG. 1 shows an embodiment of the disclosed apparatus; and

FIG. 2 shows an embodiment of a method according to the disclosed embodiments.

### DETAILED SPECIFICATION

Disclosed embodiments illustrate an apparatus and method utilizing an array 100 of at least one effector 110 capable of delivering electromagnetic fields to the brain (or other section of the central nervous system) of a human or non-human animal subject (denoted as the “subject”) promptly after an acute injury to one or more areas (said injured area denoted as a “lesion”) within a brain of the subject. For the purposes of this specification, the term “promptly” may be understood as being within an hour of the injury, or within four hours after the injury, or within 72 hours after the injury. The term “acute injury” is understood to mean an insult (for example vascular or traumatic) that has worsened or developed within a short time, for example less than one minute, or less than one hour, or less than four hours, or less than 72 hours.

The disclosed embodiments refer to prior publication by Weinberg US 20220397625, entitled, Apparatus and Method for Magnetic Resonance Imaging with Electropermanent Magnets” and US 20220212022 “Method and Apparatus for Selective Modulation of Neuronal Function and of Tissue Permeability with MRI Correlation” incorporated by reference in their entirety.

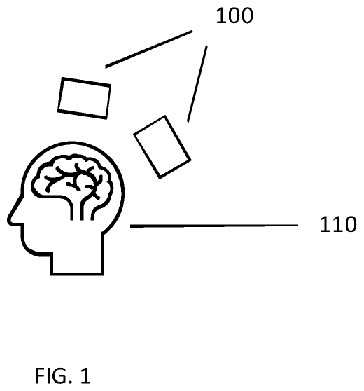

FIG. 1 shows an embodiment of the apparatus. An array of at least one electromagnetic effector is placed in proximity to a section of the nervous system of a human or non-human subject 110. The electromagnetic effector may be an electropermanent magnet, or may be an electrode. Not shown, but included in the apparatus, are the control or power circuitry or display method that are required to control or power the effector array.

An array 100 of at least one effector is in proximity to the brain of the subject 110. For the purposes of this specification, “proximity” may be understood as being within a distance that the electromagnetic field of the effector is above a certain threshold within the lesion. It is understood that the electromagnetic field may be an electric field induced by the changing magnetic field generated by the array. For the purposes of this specification, the threshold may be 10 volts per meter, 50 volts per meter, 100 volts per meter, or 200 volts per meter.

It is understood that the effector array 100 may be comprised of electropermanent magnets (“EPMs”) capable of generating and delivering (to a lesion) electrically-induced fields and/or currents, or may be comprised of electrodes for generation and delivery (to a lesion) of electric fields and/or currents, or may be comprised of electrical coils, or of a combination of these types of components. It is understood that other components may be needed for operation of the apparatus, including a computer, a display to a human user, and control and power circuitry and sensors, as have been, but are not included in FIG. 1 for the sake of clarity of illustration.

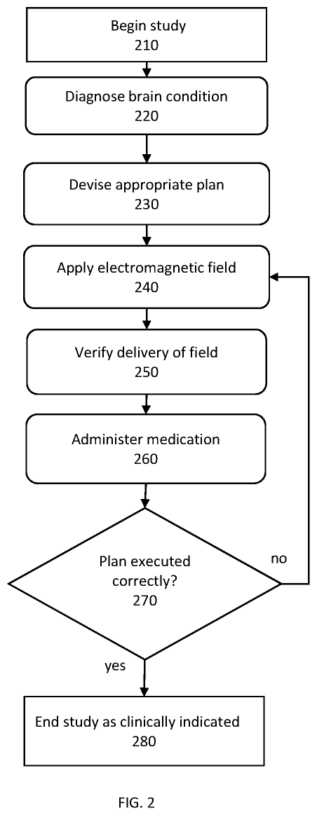

FIG. 2 shows an embodiment of the method. In operation 210, a user begins a diagnostic study and/or therapeutic procedure of the central nervous system of a subject, who may be a human or non-human animal. Based on clinical observations (for example, loss of consciousness), the subject may have had an acute injury to the brain, for example a stroke. In operation 220 the apparatus may be used to assist in the diagnosis of the acute injury, for example by collecting an image of the brain with magnetic resonance imaging using electropermanent magnets. In operation 230, a treatment plan is devised either by a medical or veterinary practitioner or by artificial intelligence or both. The treatment plan may be made on the basis of clinical observations or on the basis of the diagnostic information collected in operation 220. In operation 240, electric and/or magnetic fields may be applied by the apparatus to the central nervous system, which may be applied according to the plan of step 230. In operation 250, the extent and/or effect of the electric and/or magnetic fields applied in step 240 may be assessed, for example, by examining brain magnetization. In operation 260, a medication (drug or gene) may be administered. In operation 270, an assessment is made, either by a medical or veterinary practitioner or by artificial intelligence or both, as to whether the treatment of operation 240 was delivered according to the plan of operation 230. If the plan has been executed, the procedure may be terminated in operation 280 according to clinical requirements. For example, the subject may then begin a vascular procedure to open a vessel contributing to a stroke.

It is understood that the application of the electromagnetic field by the apparatus may open the blood-brain barrier or otherwise enhance delivery of such medication or device to the lesion or to portions of the central nervous system that are connected (e.g., via neurons) to the lesion. For example, the device may be a magnetic particle containing medications, said particle being manipulated by magnetic fields as taught by Weinberg US 2013020412 “Equipment and Methodologies for Magnetically-Assisted Delivery of Therapeutic Agents Through Barriers”

It is understood that the disclosed embodiments may be used within an ambulance or emergency department setting, for example while waiting for definitive therapy (e.g., vascular repair).

It is understood that the term “electromagnetic fields” may mean electric fields, electrical fields that are induced by changing magnetic fields, or magnetic fields, or a combination of any or all of those fields.

It is understood that the disclosed embodiments may be applied to any portion of the central nervous system of the subject. The terms “brain” and “central nervous system” are used interchangeably in this description.

It is understood that one mechanism of therapeutic action is to generate an electromagnetic field that stimulates neurons or nerves in the central nervous system either within the lesion or connected to the lesion via neuronal pathways (or both), so that said neurons are not inactive shortly after the injury. It is understood that the prompt inactivity of the nerves or neurons may lead to long-term loss of functional recovery if not stimulated (or otherwise modulated) by the electromagnetic field. It is understood that another mechanism may be to prevent non-neuron cells from destroying neurons in the short time period following the injury. It is understood that one or both of these mechanisms, or some other mechanism, may assist in promoting recovery after the injury.

For the purposes of this description, an electropermanent magnet (“EPM”) is defined as a magnetizable material and a coil, whereby the magnetizable material retains some magnetization after an electrical current has passed through the coil.

It is understood that the EPM may be one of multiple EPMs (for example, in an array of EPMs), and may be combined with permanent magnets in the same or different arrays.

It is understood that the lesion may be vascular (for example an occlusion of a vessel) or traumatic (for example from mechanical forces) or some other etiology or combination of etiologies.

It is understood that the effect on tissues generated by the effectors may be through application of electric fields to the tissues, induction of electric fields in the tissues, or through forces on magnetic particles, or through some other means.

It is understood that the combination of electromagnetic fields from an array can provide a more effective field at lesions deep in the brain, as taught by Weinberg. For the purposes of this disclosure, the term “deep” is meant to be more than 5 centimeters, or more than 10 centimeters.

It is understood that a medication may be delivered to the lesion while electromagnetic fields are applied with the invention.

In some embodiments, the prompt application of electromagnetic fields to a lesion in the central nervous system of a subject after an acute injury may help in promoting functional recovery of the subject.

Those skilled in the art will recognize, upon consideration of the above teachings, that the above exemplary embodiments and the controller may be based upon use of one or more programmed processors programmed with a suitable computer program. However, the disclosed embodiments could be implemented using hardware component equivalents such as special purpose hardware and/or dedicated processors. Similarly, general purpose computers, microprocessor based computers, micro-controllers, optical computers, analog computers, dedicated processors, application specific circuits and/or dedicated hard wired logic may be used to construct alternative equivalent embodiments.

Moreover, it should be understood that control and cooperation of the above-described components may be provided using software instructions that may be stored in a tangible, non-transitory storage device such as a non-transitory computer readable storage device storing instructions which, when executed on one or more programmed processors, carry out the above-described method operations and resulting functionality. In this case, the term “non-transitory” is intended to preclude transmitted signals and propagating waves, but not storage devices that are erasable or dependent upon power sources to retain information.

Those skilled in the art will appreciate, upon consideration of the above teachings, that the program operations and processes and associated data used to implement certain of the embodiments described above can be implemented using disc storage as well as other forms of storage devices including, but not limited to non-transitory storage media (where non-transitory is intended only to preclude propagating signals and not signals which are transitory in that they are erased by removal of power or explicit acts of erasure) such as for example Read Only Memory (ROM) devices, Random Access Memory (RAM) devices, network memory devices, optical storage elements, magnetic storage elements, magneto-optical storage elements, flash memory, core memory and/or other equivalent volatile and non-volatile storage technologies without departing from certain embodiments. Such alternative storage devices should be considered equivalents.

1. An apparatus for promoting recovery after an acute injury to the central nervous system in a subject comprising:
at least one array of at least one effector in proximity to a lesion in the central nervous system of a subject, wherein the array is configured to generate electrical or magnetic fields in the lesion promptly after acute injury and stimulate neurons or nerves in the central nervous system either within the lesion or connected to the lesion via neuronal pathways.

2. The apparatus of claim 1, wherein the effector is an electropermanent magnet.

3. The apparatus of claim 1, wherein the effector is an electrode.

4. The apparatus of claim 1, wherein the array is configured to create an image of the lesion.

5. The apparatus of claim 1, wherein the array defines an extent and magnitude of delivery of electromagnetic fields to the lesion.

5. The apparatus of claim 1, wherein the lesion is in the brain.

7. A method for promoting recovery after an acute injury to the central nervous system in a subject comprising:
generating an electromagnetic field in a lesion via an apparatus with an array of at least one effector promptly after the acute injury to stimulate neurons or nerves in the central nervous system either within the lesion or connected to the lesion via neuronal pathways.

8. The method of claim 7, wherein the electromagnetic field is applied within one hour after the acute injury.

9. The method of claim 7, wherein the electromagnetic field is applied within four hours after the acute injury.

10. The method of claim 7, wherein the electromagnetic field is applied within 72 hours after the acute injury.

11. The method of claim 7, wherein a medication is administered to the subject while the electromagnetic field is applied.

12. The method of claim 7, where an image of the lesion collected by the apparatus is used in planning the delivery of electromagnetic radiation to the lesion.

13. The method of claim 7, wherein the apparatus is used in assessing the delivery of electromagnetic fields to the lesion.

14. The method of claim 7, wherein the apparatus is used during transport of the subject.

15. The method of claim 7, wherein the apparatus delivers electromagnetic fields to deep lesions.

16. The method of claim 7, wherein the apparatus delivers magnetic particles or other magnetically-actuated devices fields to the lesion.

17. The method of claim 7, wherein the injury is vascular.

18. The method of claim 7, wherein the injury is traumatic.
